(19) United States
(12) Patent Application Publication
(10) Pub. No.: US 2025/0078991 A1
(43) Pub. Date: Mar. 6, 2025

(54) SYSTEM AND METHOD FOR DETERMINING PREDICTED ALLOCATIONS OF RESOURCES FOR A HEALTHCARE FACILITY

(71) Applicant: Steward St. Elizabeth's Medical Center, Boston, MA (US)

(72) Inventors: Ralph de la Torre, Dallas, TX (US); David Collarusso, Dallas, TX (US)

(73) Assignee: Steward St. Elizabeth's Medical Center, Boston, MA (US)

(21) Appl. No.: 18/824,633

(22) Filed: Sep. 4, 2024

Related U.S. Application Data
(60) Provisional application No. 63/536,893, filed on Sep. 6, 2023.

(51) Int. Cl. G16H 40/20

(52) U.S. Cl. CPC G16H 40/20

(57) ABSTRACT

An Artificial Intelligence (AI) based computer system and method for determining one or more predicted requirements and/or events for a healthcare facility corresponding to a scheduled inflow of patients. Generated is a learning inference model using a machine learning and/or deep learning algorithm configured to capture data, from the computer network, containing information relating to patient inflow to the healthcare facility, wherein the data includes a purpose of stay for a patient. The captured data is analyzed, using the generated learning inference model, to generate, using at least a portion of the captured data, one or more predictions regarding one or more conditions to occur in the future that are associated with one or more resources of the healthcare facility associated with the purpose of stay for the patient. The one or more predictions are then analyzed, using the generated learning inference model, for recommending an allocation of at least one of the one or more.

### CROSS REFERENCE TO RELATED APPLICATIONS

This application claims priority to U.S. Patent Application Ser. No. 63/536,893 filed Sep. 6, 2023, which is incorporated herein by reference in its entirety.

### FIELD OF THE INVENTION

The illustrated embodiments generally relate to systems, methods and apparatuses for determining and predicting the capacity and availability of various resources in healthcare facilities, and more particularly to using Artificial Intelligence techniques for providing decision support capabilities to improve resource management and planning.

### BACKGROUND OF THE INVENTION

Currently some healthcare facilities face problems associated with managing capacity and resources as well as the flow of patients through the health care facility. Thus, as the number of patients entering the healthcare system increases, the number of healthcare professionals, such as physicians, nurses, etc. to care for these patients will also likely increase. Hence, the impact of these increasing number of patients are impacting increasing strain on resource management for healthcare facilities, such as hospitals.

The capacity and resources of the health care system are affected by a number of variables. For example, baby boomers are steadily reaching an age where more intense health care is needed. There also is an increasing number of emergency room visits due to a declining percentage of the population with health care insurance. Additionally, less than ideal health care facility processes and procedures for ensuring an efficient process of moving patients throughout the healthcare facility during their stay as well as inefficient clinical processes for maintaining minimal variations in care between patients impacts the capacity and resources allocated within a health care system.

The above-mentioned problems oftentimes translate into a myriad of negative consequences for healthcare facilities. In particular, decreased profit margins typically occur as patient lengths of stay increase due to increased care demands and increased complexities in managing care. When capacity loads and patient statuses are not properly predicted, the finite healthcare resources may be unable to manage current needs in a timely or cost-effective manner.

Additionally, increased wait times and other delays may lead to a decrease in patient satisfaction and, in some cases, a decrease in the quality of care provided to patients. Moreover, inappropriately staffed care environments may lead to mistakes in care decisions and higher probabilities of time-dependent care not occurring in the needed window of time which could lead to the patient's health further deteriorating.

Furthermore, the unpredictability and turbulence of a hospital care environment with capacity and patient inflow difficulties is often detrimental to job satisfaction and retention of skilled staff such as nurses and allied care providers. Increasingly busy health care units and unpredictable patient loads often leaves health care professionals, such as for example, nurses feeling high levels of job stress and decreased abilities to control their work experience. Additionally, long wait times for medical care by patients frequently leads to overworked transport and ancillary staff, which increases employee overtime costs. These variables have been shown to decrease job satisfaction and job retention rates and may result in unsatisfied patients. Moreover, some health care facilities do not have an organized process for ensuring appropriate patient movements through the various units of the health care facility. In this regard, it may be difficult for health care facilities to understand the end-to-end patient flow throughout the enterprise and understand the causes of bottlenecks and backups in the health care system.

Thus, a need exists to provide an efficient mechanism to predict patient load and capacity for planning and allocating resources in health care facilities in a predictive manner.

### SUMMARY OF THE INVENTION

The purpose and advantages of the illustrated embodiments will be set forth in and apparent from the description that follows. Additional advantages of the illustrated embodiments will be realized and attained by the devices, systems and methods particularly pointed out in the written description and claims hereof, as well as from the appended drawings.

In accordance with a purpose of the illustrated embodiments, in one aspect, a system and method for proactively tracking the length of stay of patients in a healthcare facility by a computer system, preferably using Artificial Intelligence (AI) techniques, is described herein. In particular, the computer method and system of the illustrated embodiments relate to receiving patient flow data reflecting patient inflow to the healthcare facility, tracking the length of stay of patients in the facility, and predicting the length of stay of the patients in the facility.

In accordance with other aspects of the illustrated embodiments, provided is a mechanism in which computer systems, preferably using Artificial Intelligence (AI) techniques, utilize one or more predictive tools that incorporate flow between health care units, staffing, supplies and clinical information to allow healthcare organizations to identify in real-time, or in the near future, areas of capacity constraints, flow problems, supply (e.g., number of appropriate nurses/doctors, beds, rooms, healthcare facility supplies, operating room supplies, etc.) and demand (e.g., number of current patient) mismatches. These tools provide real-time and future visibility of capacity problems across various healthcare units in a healthcare facility.

In accordance with other aspects of the illustrated embodiments, provided is an Artificial Intelligence (AI) based computer system and method for determining one or more predicted requirements and/or events for a healthcare facility corresponding to a scheduled inflow of patients. Generated is a learning inference model using a machine learning and/or deep learning algorithm configured to capture data, from the computer network, containing information relating to patient inflow to the healthcare facility, wherein the data includes a purpose of stay for a patient. The captured data is analyzed, using the generated learning inference model, to generate, using at least a portion of the captured data, one or more predictions regarding one or more conditions to occur in the future that are associated with one or more resources of the healthcare facility associated with the purpose of stay for the patient. The one or more predictions are then analyzed, using the generated learning inference model, for recommending an allocation and/or reallocation of at least one of the one or more resources on the basis of the results. By using artificial intelligence and/or machine learning techniques, the illustrated embodiments provide significant improvements in computer applications for determining one or more predicted requirements and/or events for a healthcare facility corresponding to a scheduled inflow of patients.

### BRIEF DESCRIPTION OF THE DRAWINGS

The accompanying appendices and/or drawings illustrate various, non-limiting, examples, inventive aspects in accordance with the present disclosure:

FIG. 1 illustrates an example communication network utilized with one or more of the illustrated embodiments;

FIG. 2 illustrates an example network device/node utilized with one or more of the illustrated embodiments;

FIG. 3 illustrates a diagram depicting an Artificial Intelligence (AI) device utilized with one or more of the illustrated embodiments;

FIG. 4 illustrates a diagram depicting an AI server utilized with one or more of the illustrated embodiments;

FIG. 5 is a schematic diagram of a proactive labor management (PLM) system in accordance with an embodiment;

FIG. 6 are graphs illustrating proactive length of stay monitoring by a PLM server of the PLM system of FIG. 1;

FIG. 7 is a graph of proactive staff management by the PLM server of the PLM system of FIG. 1; and

FIG. 8 is a process diagram illustrating a process for determining one or more predicted requirements and/or events for a healthcare facility corresponding to a scheduled inflow of patients using Artificial Intelligence (AI) techniques.

### DETAILED DESCRIPTION OF CERTAIN EMBODIMENTS

The illustrated embodiments are now described more fully with reference to the accompanying drawings wherein like reference numerals identify similar structural/functional features. The illustrated embodiments are not limited in any way to what is illustrated as the illustrated embodiments described below are merely exemplary, which can be embodied in various forms, as appreciated by one skilled in the art. Therefore, it is to be understood that any structural and functional details disclosed herein are not to be interpreted as limiting, but merely as a basis for the claims and as a representation for teaching one skilled in the art to variously employ the discussed embodiments. Furthermore, the terms and phrases used herein are not intended to be limiting but rather to provide an understandable description of the illustrated embodiments.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the illustrated embodiments, exemplary methods and materials are now described.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

It is to be appreciated the illustrated embodiments discussed below are preferably a software algorithm, program or code residing on computer useable medium having control logic for enabling execution on a machine having a computer processor. The machine typically includes memory storage configured to provide output from execution of the computer algorithm or program.

As used herein, the term “software” is meant to be synonymous with any code or program that can be in a processor of a host computer, regardless of whether the implementation is in hardware, firmware or as a software computer product available on a disc, a memory storage device, or for download from a remote machine. The embodiments described herein include such software to implement the equations, relationships and algorithms described above. One skilled in the art will appreciate further features and advantages of the illustrated embodiments based on the above-described embodiments. Accordingly, the illustrated embodiments are not to be limited by what has been particularly shown and described, except as indicated by the appended claims.

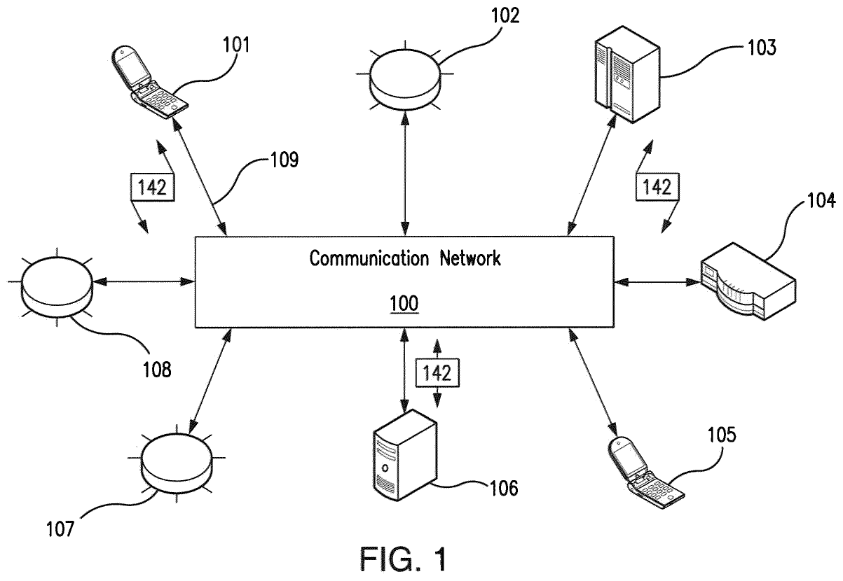

Turning now descriptively to the drawings, in which similar reference characters denote similar elements throughout the several views, FIG. 1 depicts an exemplary communications network 100 in which below illustrated embodiments may be implemented. It is to be understood a communication network 100 is a geographically distributed collection of nodes interconnected by communication links and segments for transporting data between end nodes, such as personal computers, work stations, smart phone devices, tablets, televisions, sensors and or other devices such as automobiles, etc. Many types of networks are available, with the types ranging from local area networks (LANs) to wide area networks (WANs). LANs typically connect the nodes over dedicated private communications links located in the same general physical location, such as a building or campus. WANs, on the other hand, typically connect geographically dispersed nodes over long-distance communications links, such as common carrier telephone lines, optical lightpaths, synchronous optical networks (SONET), synchronous digital hierarchy (SDH) links, or Powerline Communications (PLC), and others.

FIG. 1 is a schematic block diagram of an example communication network 100 illustratively comprising nodes/devices 101-108 (e.g., sensors 102, client computing devices 103, smart phone devices 105, web servers 106, routers 107, switches 108, databases, and the like) interconnected by various methods of communication. For instance, the links 109 may be wired links or may comprise a wireless communication medium, where certain nodes are in communication with other nodes, e.g., based on distance, signal strength, current operational status, location, etc. Moreover, each of the devices can communicate data packets (or frames) 142 with other devices using predefined network communication protocols as will be appreciated by those skilled in the art, such as various wired protocols and wireless protocols etc., where appropriate. In this context, a protocol consists of a set of rules defining how the nodes interact with each other. Those skilled in the art will understand that any number of nodes, devices, links, etc. may be used in the computer network, and that the view shown herein is for simplicity. Also, while the embodiments are shown herein with reference to a general network cloud, the description herein is not so limited, and may be applied to networks that are hardwired.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the illustrated embodiments are described below with reference to flowchart illustrations and/or block diagrams of methods, apparatus (systems) and computer program products according to the illustrated embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

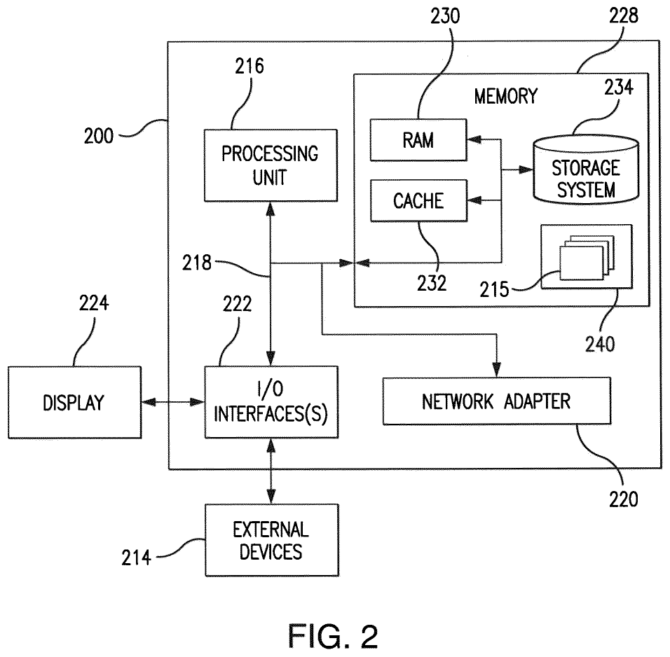

FIG. 2 is a schematic block diagram of an example network computing device 200 (e.g., client computing device 103, server 106, etc.) that may be used (or components thereof) with one or more embodiments described herein, e.g., as one of the nodes shown in the network 100. As explained above, in different embodiments these various devices are configured to communicate with each other in any suitable way, such as, for example, via communication network 100.

Device 200 is intended to represent any type of computer system capable of carrying out the teachings of various illustrated embodiments. Device 200 is only one example of a suitable system and is not intended to suggest any limitation as to the scope of use or functionality of the illustrated embodiments described herein. Regardless, computing device 200 is capable of being implemented and/or performing any of the functionality set forth herein.

Computing device 200 is operational with numerous other general purpose or special purpose computing system environments or configurations. Examples of well-known computing systems, environments, and/or configurations that may be suitable for use with computing device 200 include, but are not limited to, personal computer systems, server computer systems, thin clients, thick clients, hand-held or laptop devices, multiprocessor systems, microprocessor-based systems, network PCs, minicomputer systems, and distributed data processing environments that include any of the above systems or devices, and the like. Computing device 200 may be described in the general context of computer system-executable instructions, such as program modules, being executed by a computer system. Generally, program modules may include routines, programs, objects, components, logic, data structures, and so on that perform particular tasks or implement particular abstract data types. Computing device 200 may be practiced in distributed data processing environments where tasks are performed by remote processing devices that are linked through a communications network. In a distributed data processing environment, program modules may be located in both local and remote computer system storage media including memory storage devices.

The components of device 200 may include, but are not limited to, one or more processors or processing units 216, a system memory 228, and a bus 218 that couples various system components including system memory 228 to processor 216. Bus 218 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. Computing device 200 typically includes a variety of computer system readable media. Such media may be any available media that is accessible by device 200, and it includes both volatile and non-volatile media, removable and non-removable media.

System memory 228 can include computer system readable media in the form of volatile memory, such as random-access memory (RAM) 230 and/or cache memory 232. Computing device 200 may further include other removable/non-removable, volatile/non-volatile computer system storage media. By way of example only, storage system 234 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 218 by one or more data media interfaces. As will be further depicted and described below, memory 228 may include at least one program product having a set (e.g., at least one) of program modules that are configured to carry out the functions of embodiments of illustrated embodiments.

Program/utility 240, having a set (at least one) of program modules 215, such as underwriting module, may be stored in memory 228 by way of example, and not limitation, as well as an operating system, one or more application programs, other program modules, and program data. Each of the operating system, one or more application programs, other program modules, and program data or some combination thereof, may include an implementation of a networking environment. Program modules 215 generally carry out the functions and/or methodologies of the illustrated embodiments as described herein.

Device 200 may also communicate with one or more external devices 214 such as a keyboard, a pointing device, a display 224, etc.; one or more devices that enable a user to interact with computing device 200; and/or any devices (e.g., network card, modem, etc.) that enable computing device 200 to communicate with one or more other computing devices. Such communication can occur via Input/Output (I/O) interfaces 222. Still yet, device 200 can communicate with one or more networks such as a local area network (LAN), a general wide area network (WAN), and/or a public network (e.g., the Internet) via network adapter 220. As depicted, network adapter 220 communicates with the other components of computing device 200 via bus 218. It should be understood that although not shown, other hardware and/or software components could be used in conjunction with device 200. Examples, include, but are not limited to: microcode, device drivers, redundant processing units, external disk drive arrays, RAID systems, tape drives, and data archival storage systems, etc.

FIGS. 1 and 2 are intended to provide a brief, general description of an illustrative and/or suitable exemplary environment in which the below described illustrated embodiments may be implemented. FIGS. 1 and 2 are exemplary of a suitable environment and are not intended to suggest any limitation as to the structure, scope of use, or functionality of an illustrated embodiment. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

It is to be understood the embodiments described herein are preferably provided with self-learning/Artificial Intelligence (AI) for determining one or more predicted requirements events for a healthcare facility corresponding to a scheduled inflow of patients as described below in accordance with the illustrated embodiments. The computer system 200 is integrated with an AI system (as also described below) that is coupled to a plurality of external databases/data sources that implements machine learning and artificial intelligence algorithms in accordance with the illustrated embodiments. For instance, the AI system may include two subsystems: a first sub-system that learns from historical data; and a second subsystem to identify and recommend one or more parameters or approaches based on the learning. It should be appreciated that although the AI system may be described as two distinct subsystems, the AI system can also be implemented as a single system incorporating the functions and features described with respect to both subsystems.

In accordance with the illustrated embodiments described herein, artificial intelligence refers to the field of studying artificial intelligence or methodology for making artificial intelligence, and machine learning refers to the field of defining various issues dealt with in the field of artificial intelligence and studying methodology for solving the various issues. Machine learning is defined as an algorithm that enhances the performance of a certain task through a steady experience with the certain task.

Also in accordance with the illustrated embodiments, an artificial neural network (ANN) is a model used in machine learning and may mean a whole model of problem-solving ability which is composed of artificial neurons (nodes) that form a network by synaptic connections. The artificial neural network can be defined by a connection pattern between neurons in different layers, a learning process for updating model parameters, and an activation function for generating an output value. The artificial neural network may include an input layer, an output layer, and optionally one or more hidden layers. Each layer includes one or more neurons, and the artificial neural network may include a synapse that links neurons to neurons. In the artificial neural network, each neuron may output the function value of the activation function for input signals, weights, and deflections input through the synapse.

Model parameters refer to parameters determined through learning and include a weight value of synaptic connection and deflection of neurons. A hyperparameter means a parameter to be set in the machine learning algorithm before learning, and includes a learning rate, a repetition number, a mini batch size, and an initialization function. The purpose of the learning of the artificial neural network may be to determine the model parameters that minimize a loss function. The loss function may be used as an index to determine optimal model parameters in the learning process of the artificial neural network. Machine learning may be classified into supervised learning, unsupervised learning, and reinforcement learning according to a learning method. The supervised learning may refer to a method of learning an artificial neural network in a state in which a label for learning data is given, and the label may mean the correct answer (or result value) that the artificial neural network must infer when the learning data is input to the artificial neural network. The unsupervised learning may refer to a method of learning an artificial neural network in a state in which a label for learning data is not given. The reinforcement learning may refer to a learning method in which an agent defined in a certain environment learns to select a behavior or a behavior sequence that maximizes cumulative compensation in each state.

Machine learning, which is implemented as a deep neural network (DNN) including a plurality of hidden layers among artificial neural networks, is also referred to as deep learning, and the deep learning is part of machine learning.

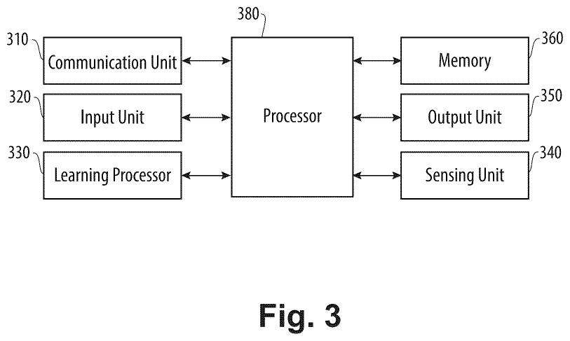

FIG. 3 illustrates an AI device 300 according to an embodiment of the present invention, which as described herein, is configured and operative to determine one or more predicted requirements and/or events for a healthcare facility corresponding to a scheduled inflow of patients.

The AI device 300 may be implemented by a stationary device or a mobile device, such as a web server, a desktop computer, a notebook, a desktop computer, and the like.

Referring to now FIG. 3, in conjunction with FIGS. 1 and 2, the AI device 300 is operatively coupled to, or integrated with computing device 200, in accordance with the illustrated embodiments described herein. AI device 300 preferably includes a communication unit 310, an input unit 320, a learning processor 330, a sensing unit 340, an output unit 350, a memory 360, and a processor 380. The communication unit 310 may transmit and receive data to and from external devices, such as other AI devices, by using wire/wireless communication technology. For example, the communication unit 310 may transmit and receive sensor information, a user input, a learning model, and a control signal to and from external devices, such as AI server 400.

The communication technology used by the communication unit 310 preferably includes GSM (Global System for Mobile communication), CDMA (Code Division Multi Access), LTE (Long Term Evolution), 5G, WLAN (Wireless LAN), Wi-Fi (Wireless-Fidelity), Bluetooth™, RFID (Radio Frequency Identification), Infrared Data Association (IrDA), ZigBee, NFC (Near Field Communication), and the like.

The input unit 320 may acquire various kinds of data, including, but not limited to patient information and healthcare related data. The input unit 320 may acquire a learning data for model learning and an input data to be used when an output is acquired by using a learning model. The input unit 320 may acquire raw input data. In this case, the processor 380 or the learning processor 330 may extract an input feature by preprocessing the input data. The learning processor 330 may learn a model composed of an artificial neural network by using learning data. The learned artificial neural network may be referred to as a learning model. The learning model may be used to an infer result value for new input data rather than learning data, and the inferred value may be used as a basis for determination to perform a certain operation.

At this time, the learning processor 330 may perform AI processing together with the learning processor 440 of the AI server 400, and the learning processor 330 may include a memory integrated or implemented in the AI device 300. Alternatively, the learning processor 330 may be implemented by using the memory 360, an external memory directly connected to the AI device 300, or a memory held in an external device. The sensing unit 340 may acquire at least one of internal information about the AI device 300, ambient environment information about the AI device 300, and user information by using various sensors.

The output unit 350 preferably includes a display unit for outputting/displaying relevant information to a user in accordance with the illustrated embodiments described herein. The memory 360 preferably stores data that supports various functions of the AI device 300. For example, the memory 360 may store input data acquired by the input unit 320, learning data, a learning model, a learning history, and the like.

The processor 380 preferably determines at least one executable operation of the AI device 300 based on information determined or generated by using a data analysis algorithm or a machine learning algorithm. The processor 380 may control the components of the AI device 300 to execute the determined operation. To this end, the processor 380 may request, search, receive, or utilize data of the learning processor 330 or the memory 360. The processor 380 may control the components of the AI device 300 to execute the predicted operation or the operation determined to be desirable among the at least one executable operation. When the connection of an external device is required to perform a determined operation, the processor 380 may generate a control signal for controlling the external device and may transmit the generated control signal to the external device. The processor 380 may acquire intention information for the user input and may determine the user's requirements based on the acquired intention information.

The processor 380 may acquire the intention information corresponding to the user input by using at least one of a speech to text (STT) engine for converting speech input into a text string or a natural language processing (NLP) engine for acquiring intention information of a natural language.

At least one of the STT engine or the NLP engine may be configured as an artificial neural network, at least part of which is learned according to the machine learning algorithm. At least one of the STT engine or the NLP engine may be learned by the learning processor 330, may be learned by the learning processor 340 of the AI server 400, or may be learned by their distributed processing. The processor 380 may collect history information including the operation contents of the AI device 300 or the user's feedback on the operation and may store the collected history information in the memory 360 or the learning processor 330 or transmit the collected history information to the external device such as the AI server 400. The collected history information may be used to update the learning model.

The processor 380 may control at least part of the components of AI device 300 so as to drive an application program stored in memory 360. Furthermore, the processor 380 may operate two or more of the components included in the AI device 300 in combination so as to drive the application program.

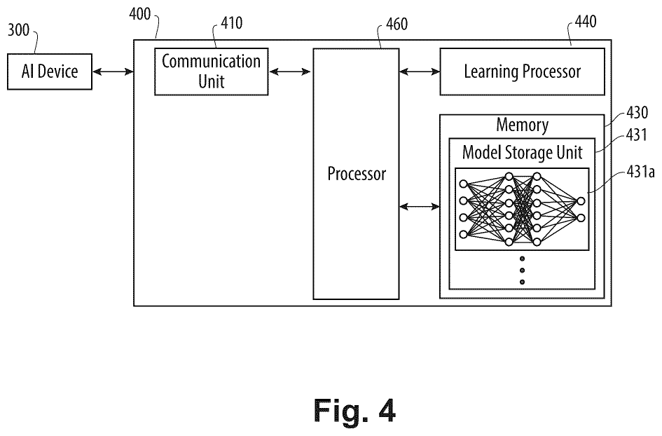

FIG. 4 illustrates an AI server 400 according to the illustrated embodiments. It is to be appreciated that the AI server 400 may refer to a device that learns an artificial neural network by using a machine learning algorithm or uses a learned artificial neural network. The AI server 400 may include a plurality of servers to perform distributed processing, or may be defined as a 5G network. At this time, the AI server 400 may be included as a partial configuration of the AI device 300, and may perform at least part of the AI processing together. The AI server 400 may include a communication unit 410, a memory 430, a learning processor 440, a processor 460, and the like. The communication unit 410 can transmit and receive data to and from an external device such as the AI device 300. The memory 430 may include a model storage unit 431. The model storage unit 431 may store a learning or learned model (or an artificial neural network 431a) through the learning processor 440. In accordance with the illustrated embodiments, the AI server is configured and operative to determine one or more predicted requirements and/or events for a healthcare facility corresponding to a scheduled inflow of patients

The learning processor 440 may learn the artificial neural network 431a by using the learning data. The learning model may be used in a state of being mounted on the AI server 400 of the artificial neural network or may be used in a state of being mounted on an external device such as the AI device 300. The learning model may be implemented in hardware, software, or a combination of hardware and software. If all or part of the learning models are implemented in software, one or more instructions that constitute the learning model may be stored in memory 430. The processor 460 may infer the result value for new input data by using the learning model and may generate a response or a control command based on the inferred result value.

With the exemplary communication network 100 (FIG. 1), computing device 200 (FIG. 2), AI device 300 (FIG. 3) and AI server 400 (FIG. 4) being generally shown and discussed above, description of certain illustrated embodiments will now be provided.

It is to be understood and appreciated that exemplary embodiments implementing one or more components of FIGS. 1-4 relate to an Artificial Intelligence (AI) based computer system and method for determining one or more predicted requirements and/or events for a healthcare facility corresponding to a scheduled inflow of patients. It is to be understood and appreciated that FIGS. 1-4 are intended to provide a brief, general description of an illustrative and/or suitable exemplary environment in which the below described illustrated embodiments may be implemented. FIGS. 1-4 are exemplary of a suitable environment and are not intended to suggest any limitation as to the structure, scope of use, or functionality of an illustrated embodiment. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

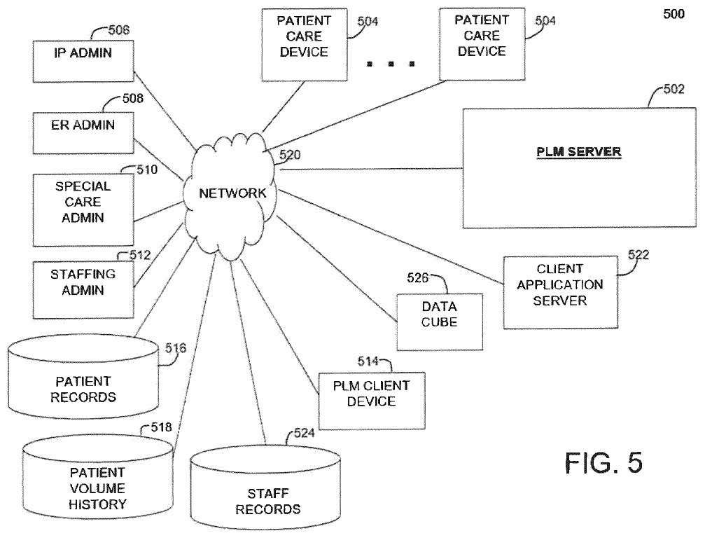

With the exemplary communication network 100 (FIG. 1), computing device 200 (FIG. 2), AI device 300 (FIG. 3) and AI server 400 (FIG. 4) being generally shown and discussed above, description of certain illustrated embodiments will now be provided. With reference now to the illustrated embodiment of FIG. 5, shown is an exemplary proactive labor management (PLM) system 500, utilizing one or more of the aforementioned communication network 100 (FIG. 1), computing device 200 (FIG. 2), AI device 300 (FIG. 3) and AI server 400 (FIG. 4), depicting one or more illustrated embodiments. As generally shown, the PLM server 502 receives patient flow data at least twice a day from a client application server 522 and one or more updated repositories. The client application server 522 and the repositories are updated by one or more computing devices, such as patient care computing devices 504, an in-patient (IP) administrative (admin) computing device 506, an emergency room (ER) admin computing device 508, a special care admin computing device 510, a staffing admin computing device 512, and/or a PLM client computing device 514. The repositories include, for example and without limitation, a patient records database 516 that stores patient medical and demographic information, a patient volume history database 518 that stores histories of patient volume relative to time with related information, and a staff records database 524 that stores staff placement and scheduling information. The PLM server 502 further receives data from a data cube 526 that obtains information that is relevant to prediction of patient flow patterns and volumes. The relevant information can include, for example and without limitation, news about weather, local events, and disease control (e.g., from the Center for Disease Control (CDC)). In an embodiment, the PLM server 502 can receive the patient flow data from the client application server 522, the repositories 516 and 524, and/or the data cube 526 on an hourly basis or on a more frequent basis.

The computing devices 504-514 can be stationary or mobile computing devices that gather data relevant to patient flow data and transmit this data to the client application server that feeds data to the PLM server 502 via at least one network 520, such as the Internet, a wide area network (WAN), or a local area network (LAN). The computing devices 504-514, the client application server 522, cube 526, and the PLM server 502 can communicate with the network 520 via one or more wired and/or wireless links. The computing devices 504-514 can gather data by receiving information from another computing device and/or via user input. PLM client computing device 514 can be dedicated to performing functions related to the PLM server 502, including displaying output from the PLM server 502.

The patient care computing devices 504 can be devices used by medical practitioners, such as doctors, nurses, and therapists. The practitioners may enter information into the patient care computing device 504 as they tend patients, and thus the information is updated frequently, such as per patient visit. Relevant information that a medical practitioner may enter includes patient arrival or discharge from a bed, scheduled procedures, patient health status, and changes in patient health status.

The IP admin computing device 506 can be operated by an IP administrative user, such as in a nurse's station on a patient floor or an admissions office that handles patient discharges, scheduled admissions or admissions transferred from another facility or department, such as the emergency room or an operating room. The IP administrative user can also enter information about patient bed assignments. This information may also be entered frequently as actions occur that are related to the patient's length of stay.

The ER admin computing device 508 can be operated by an ER administrative user that handles patient arrival at the ER (e.g., triage or assigning a bed) and enters information as actions occur that are related to the patient's length of stay. The special care admin computing device 510 can be operated be a special care medical practitioner, such as a radiologist, a respiratory therapist, a physical therapist, a social worker, a lab technician, etc., that can input information related to patient flow events as they are happening.

The patient flow data can include data about events related to the patient that are related to the patient's length of stay in a healthcare facility, including dates and times that the events are scheduled for and/or actually took place. The healthcare facility can be, for example, an in-patient or out-patient facility, such as a hospital, rehabilitation center, out-patient surgical center, etc. Examples of events for in-patient (IP) care can include: patient arrives on IP floor, target discharge date, post-acute disposition entered, patient discharge order written, patient leaves floor, and bed ready for occupancy. Examples of events for emergency room (ER) patient care can include: patient arrives in the ER, patient in triage, patient in ER bed, ER doctor assigned to patient, ER doctor places first consult or test order, request to admit to IP bed submitted, IP bed assigned, admit order is entered, and patient leaves ER.

The PLM server 502 processes the patient flow data to track status of each patient. The patient flow data can be compared to milestone data related to the patient's length of stay. Milestone data describes expected events with target times (date/time) related to a patient's length of stay. Milestones related to in-patient care can include, for example, patient arrives on IP floor (e.g., for scheduled visit, post a surgical procedure, post an ER visit), target discharge date, submission of post-acute disposition, patient discharge order submitted, patient leaves floor, bed ready for occupancy. Milestones related to ER patient care can include, for example, patient arrives in the ER (post ambulance transport), patient in triage, patient in ER bed, ER doctor assigned to patient, ER doctor places first consult or test order, request to admit to IP bed requested, IP bed assigned, admit order entered, patient leaves ER.

The target discharge date can be determined, for example, using medical guidelines and medical insurance requirements for length of stay (LOS). Patient co-morbidities can be taken into account when determining a target discharge date. Predictive analytics can be applied to determine the LOS. The predictive analytics can use information, for example and without limitation, patient demographics, historical trends, and the application of a prioritization scheme for in-house events that drive LOS. The prioritization scheme can be displayed by a patient care computing device 504 with a prioritization and/or schedule of these in-house events, such as diagnostic tests, lab tests, communicating lab results, and patient transport. The target discharge date can further be determined based on factors such as scheduling of interim milestones (e.g., a medical procedure) and patient medical status.

The client application server 522 executes client applications, such as workforce management software, accounting software, patient healthcare management software, etc. The client application server 522 receives input to the client applications from the computing devices 504-514, updates data processed by the client applications, processes the updated data using the client applications, and outputs data that can be used by the PLM server 502.

The data cube 526 can access data available on the Internet or one or more networks that is relevant to patient flow and patient volume. The data cube 526 can store and analyze the accessed data and output relevant information autonomously or in response to a query.

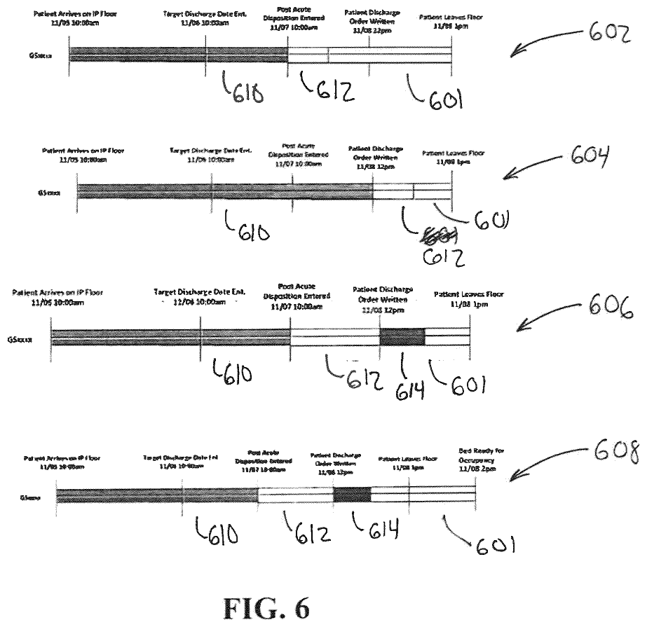

FIG. 6 shows instances of an example graph generated by the PLM server 502 that can be displayed on a computing device, such as computing devices 504-514. In the example, bar graphs 602, 604, 606, and 608 track patient flow data relative to milestones. A bar 601 advances towards one or more milestones as the patient flow data indicates that events take place. FIG. 6 shows an example of an in-patient patient, with each of the bar graphs 602-608 showing different stages or scenarios of the patient's stay. Similar graphs can be used for ER patients that track progress towards upcoming milestones. The patient in the example is associated with the following facts:

The patient has a target LOS (TLOS) of <48 hours. The following times are entered into graphs 602, 604, 606, 608 based on default rules:
Post-Acute Disposition (required before discharge)=9 AM on target discharge date
MD order written=12 PM on target discharge date
Target Discharge time=1 PM on target discharge date
Bed Ready for Occupancy=2 PM on target discharge date

Graphical indicators, such as color, are used to indicate progress in reaching milestones. A first indicator 610 (e.g., the color green) indicates the patient is on time for the next milestone's target date/time. A second indicator 612 (e.g., yellow) indicates the patient is within a threshold time (e.g., two hours) of an upcoming milestone target date/time, and has surpassed a warning threshold. A third indicator 614 (e.g., red) indicates the patient is overdue for the next milestone target date/time. An indicator is not applied to future milestones (e.g., they are not colored). Once the milestone is met, the entire bar 601 is displayed using the first indicator 610 (e.g., the bar turns green) until the patient has reached the warning threshold for the subsequent milestone.

Graph 602 is shown on November 8 at 11 AM. Graph 604 is shown on November 8 at 12:30 PM as if a discharge order was written. Graph 606 is shown on November 8 at 12:30 PM as if the discharge order was not written. Graph 608 is shown on November 8 at 12:30 PM as if the discharge order was not written and a bed ready for occupancy timestamp was included.

The PLM server 502 can further use the patient flow data to determine and update actual and/or expected patient inflow, patient outflow, and patient volume. The PLM server 502 can further process the patient flow data to determine patient volume, including actual and expected changes in patient volume.

Patient inflow can be indicated, for example, by admission of all patients to a healthcare facility (or a particular section (e.g., department), or a particular collection of healthcare facilities) or entry to the ER. Computing devices 504-510 can provide information about, for example, admissions related to elective procedures, emergency situations (e.g., from the ER), transfers from other healthcare facilities, etc. For example, the patient inflow data received from the computing devices 504-510 can include hourly admission census data, daily admission census data, multiple times daily (e.g., three times) pharmacy data, and billing data (e.g., daily reports.

The PLM server 502 can further store patient inflow data in the patient volume history database 518 as historical patient inflow data. The PLM server 502 can further analyze the historical patient inflow data for patterns related to time of day, day of the week, time of the month, time of the year (e.g., seasons, holidays), source of admission, etc. The analysis can be used to predict patient inflow as related to time of day, day of the week, time of the month, time of the year, source of admission, etc.

Patient outflow can be indicated by patient discharge from the healthcare facility or ER, as provided by computing devices 504-510. The PLM server 502 can further store patient outflow data and LOS data in the patient volume history database 518 as historical patient outflow data and LOS. The PLM server 502 can further analyze the historical patient outflow data and LOS data for patterns related to time of day, day of the week, time of the month, time of the year, seasons, holidays, source of admission, etc.

On an individual basis, the patient discharge data can be predicted for a patient by predicting LOS based on the patient's demographics (e.g., age, gender, language, weight) and medical information (e.g., DRG, co-morbidities, attending physician). The patient's target discharge date can be compared to a target LOS (TLOS). The TLOS can be determined based on, in addition to medical guidelines and health insurance requirements related to the patient's DRG, historical LOS information for patients with similar medical conditions (e.g., historical information for patients (e.g. DRG history) and similar demographics. Patients at risk for a high negative variance in LOS, which refers to when a TLOS is predicted to be significantly later than a target discharge date, can be flagged for closer hospital management.

On a patient population basis for patients admitted or predicted to be admitted, TLOS and target discharge dates, as well as historical discharge and LOS patterns, can be used to predict discharge dates. The historical discharge patterns and LOS patterns may indicate that the time of day, day of the week, time of month, or time of year (e.g., season or holiday) can influence the LOS and the discharge date.

The patient volume is determined by the patient inflow and the patient outflow. The patient inflow can include any patients already staying at the healthcare facility as of the time that tracking was begun. The patient volume at any given time indicates the number of patients staying at the healthcare facility at that time. The patient flow data is updated and processed by the PLM server 502 at least twice a day, (e.g., AM/PM, per shift, or hourly). In an embodiment, the patient flow data is updated and processed by the PLM server 502 on an hourly basis. The PLM server 502 can further store patient volume data in the patient volume history database 518 as historical patient volume data.

The PLM server 502 can access historical patient inflow, outflow, LOS, and volume data stored in the history database 118 to detect trends and use detected trends to make predictions about individual patient flow, patient inflow, patient outflow, and/or patient volume of a patient population in a healthcare facility, a section (e.g., one or more units, departments, etc.) of a healthcare facility, or a collection of one or more healthcare facilities (e.g., that are affiliated or owned or managed by a common entity). The PLM server 502 can further use the predictions about patient flow and/or volume to determine staffing needs and manage staffing. The predictions about patient flow and/or volume can be based on one or more levels of granularity of time, such as hourly, per shift, at least twice a day, daily, per day of the week, monthly, seasonally, per calendar events (e.g., holidays), and/or yearly. Additionally, the historical patient inflow, outflow, LOS, and volume data can indicate short term or long term trends that can further be used to determine and manage staffing needs.

The patient inflow and outflow data can be received from the computing devices 504-510 in near real time to reflect patient flow events as they are recorded and transmitted. The patient inflow, outflow, and volume data can be analyzed for immediate, short term (e.g., 0-5 days), long term, or retrospective reporting and intercedence purposes. Reporting can include expected or actual (if known) billing value. Real time reporting can show real time patient volumes by time of day, or unit. This can be updated at regular intervals, such as every 15 minutes. Reporting can show budget variances, expected volume, origin of patient inflow, and outflow destination. Reporting can also recognize and project hourly, weekly, seasonal, holiday, service mix (e.g., ER, Medical/surgical, ICU), and macro event (e.g., flu vaccine effectiveness, aging baby boomer population) impact trends. Retrospective reporting about volume data can highlight missed opportunities to reduce staffing. The reports can be used to predict needs for future patient placement or staffing.

The staffing admin computing device 512, receives (from another computing device or by user input) staffing data describing the actual and planned volume and makeup of the staffing, and provides the staffing data to the client application server 522. The client application server 522 can store updated data about staff placement and staff schedules in the staff records database 524. The client application server 522 can further provide staffing updates about the staff placement and staff schedules to the PLM server 502. Staffing updates provided to the PLM server 502 can be provided at regular intervals or on demand. In an embodiment, the staffing updates are provided at least twice a day, and in an embodiment, the staffing updates are provided hourly or more frequently. Certain staffing data can be provided on a daily basis. Payroll information can be provided on a payroll schedule basis, e.g., biweekly. The staffing data can describe information about staffing actual and planned staffing volume on an hourly basis, including the hourly cost per staff member, and how various staff types are distributed across departments and scheduled across shifts. The staffing data can be stored in the staff records database 524 and aggregated by selectable categories. The categories can include, for example: department, job type, pay grade, full time, part time, overtime, flexible staff (e.g., staff that works per diem or in floating pools), shift type, which hours are cancellable, whether transferable, and productivity level, and associated costs.

The PLM server 502 integrates the patient inflow, outflow, LOS, revenue, and volume data with the staffing data. In addition, the PLM server 502 can access the patient history volume database 518 to detect patterns and trends and make predictions. The PLM server 502 can apply union rules and hospital rules to indicate opportunities to adjust staffing. The opportunities indicated can include opportunities to immediately manage a current or imminent imbalance of staffing and patient volumes. The opportunities indicated can also include opportunities for future actions, including planning or scheduling a next hiring or staff reduction event and planning for long term staff volume management.

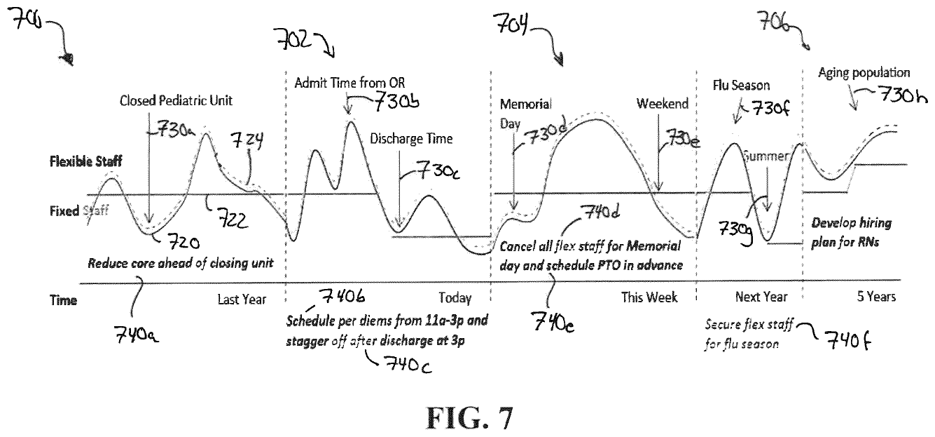

FIG. 7 shows graph 700, 702, 704, and 706 that each include plot 720, 722, and 724 that represents tracked input to PLM server 502. Plot 720 tracks patient volume over time, plot 722 tracks fixed staff volume over time, and plot 724 tracks flexible staff volume over time. In graph 700, the data plotted corresponds to the previous year. The PLM sever 502 recognizes an event 730a that is correlated to closing the pediatric unit. The PLM server 502 generates a recommendation 740a to reduce core staff ahead of closing a unit. The retrospective perspective can be used to highlight operational opportunities such as frequency of sick calls, frequency of clocking in early or late e.g., by staff member or department; report on employee utilization; and report on hiring and turnover trends.

In graph 702, the data plotted corresponds to a single day of operation. The PLM server 502 recognizes an event 730b that shows a surge in patient volume that corresponds to admit time from the operating room, and an event 730c that shows a dip in patient volume that corresponds to discharge time. The PLM server 502 can flag an opportunity 740c,d to schedule per diem staff from 11 am-3 PM and to stagger dismissal of the per diem staff after discharge at 3 PM. The daily view can be used to flag and notify hospital leadership of overstaffed situations, with a recommendation to cancel the most costly staff. Key performance indicators (KPIs) can be displayed that are targeted by user type (e.g., cost, productivity, etc.) with management and executive views. Billing revenue and staff costs can be used to display actual variances relative to the budget.

In graph 704, the data plotted corresponds to a single week of operation. The PLM server 102 recognizes an event 730d that shows a drop in patient volume that corresponds to Memorial Day, and an event 730e that shows another dip in patient volume that corresponds to the weekend. The PLM server 502 identifies and/or flags opportunities 740d,e to cancel all flexible staff for Memorial Day and schedule personal time off in advance. The weekly view can be used to optimize schedule changes (i.e., to add or cancel scheduled staff), and to flag high cost shifts (e.g., shifts that use a large amount of over-time) and identifies and/or flags the opportunity for less expensive staffing options.

In graph 706, the data plotted corresponds to a five year projection of operation. The PLM server 502 predicts an event 730f that shows surge in patient volume that corresponds to flu season, and an event 730g that shows a dip in patient volume that corresponds to summertime, and an event 730h that shows a rise in patient volume that corresponds to an aging population of baby boomers. The PLM server 502 identifies and/or flags opportunities 740f,g,h to secure flexible staff for flu season and to develop a hiring plan for nursing staff. The long term view can be used to adjust hiring and terminating of fixed staff to anticipated patient volume changes, and to adjust the flexible staff to anticipated patient volume changes to optimize staffing cost.

Thus, patient volume is actively monitored by receiving updates from computing devices 504-510 multiple times a day, such as each hour, or more frequently. The patient volume is actively analyzed to detect patterns and trends related to patient length of stay, effects of staff volume on patient's length of stay, time of day, day of the week, time of the month, time of the year (e.g., seasons, holidays), several years, type of department or facility, and source of admission. The analysis can use, for example, business logic, e.g., based on healthcare facility rules or union rules, shift types, cost factors. The PLM server 502 can use the detected patterns and trends to make predictions and to flag recommendations. The recommendations can be short term recommendations to be implemented relatively quickly, such as to cancel a scheduled shift, transfer staff, or decide on patient placement. The recommendations can be long term recommendations that can be implemented over a longer period of time, such as for scheduling hiring, training, or terminating staff.

Labor and staffing data that can be sourced from the staffing admin computing device 512 can include hourly files, daily files, weekly or biweekly payroll files, cost for all staff for the selected location and interval (total and/or broken down to groups or individuals), productivity relative to a target (total and/or broken down to groups or individuals).

KPIs about patient volume, staffing volume, and their ratios can be provided by category, with the ability to drill down through hierarchies or filter by categories. Categories can include facility, unit type, unit, and job type. KPI analytics can look at selected time intervals and time ranges. Time intervals can be, for example, hourly, daily, shift, weekly, bi-weekly, monthly, year-to-date, yearly, staffing cycle, or a rolling 12-month period. The analysis can consider, for example, number of active patient encounters, number of staff hours worked, number of staff hours paid, number of staff hours required to meet union requirements, delta of actual staff hours worked vs staff hours required by union rules, cost of all staff hours worked, number of staff hours that could have been cancelled based on union rules, cost of cancellable hours, number of staff hours that could have been transferred based on business/union rules, and cost of transferable hours. The analysis can further consider staff performance based on job type, shift type, and whether it is cancellable, transferable, or productive. The analysis can allow user to view and compare a selected cycle to other cycles or cycle averages.

The PLM server 502 can also generate display screens that users can use to implement recommendations. For example, a screen that recommends changes in patient placement can present current and projected patient volume levels at individual units, alongside unit staffing volumes to allow users to determine the best unit at which to place or transfer patients.

Unit availability information can be displayed with information about current bed availability and staff to patient ratios. For each unit, the display can include unit name, patient capacity, number of beds ready, number of patients discharging today, number of patients currently in the unit, number of staff currently required in the unit by union rules, number of staff currently in the unit, and delta of actual staff in unit to union required staff. The data can be updated regularly, e.g., every 5 (or less) minutes.

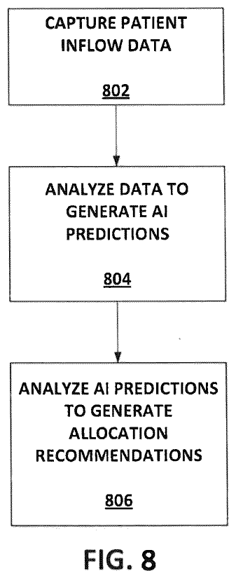

In accordance with other illustrated embodiments, and with reference to FIG. 8 (and with continuing reference to FIGS. 1-7) described is a system and method that utilizes Artificial Intelligence (AI) techniques for determining one or more predicted requirements and/or events for a healthcare facility corresponding to a scheduled inflow of patients, depicted generally by process 800 of FIG. 8. It is to be understood and appreciated that process 800 utilizes one or more systems and components described above with reference to FIGS. 1-7.

Starting at step 802, the PLM server 502 preferably generates a learning inference model using a machine learning or deep learning algorithm to capture data, from a computer network, containing information relating to patient inflow to the healthcare facility wherein the captured data includes a purpose of stay for a patient and further preferably includes information relating to the patient, wherein the purpose of stay relates to treatment of a medical condition. In certain embodiments, the captured data further includes medical history information associated with the patient, wherein at least a portion of the information relating to the patient is captured from one or more external data sources relative to the PLM server 502.

Preferably, and as mentioned above, the PLM server 502 is configured to track the length of stay of patients in the healthcare facility at least hourly and predict patient length of stay in the healthcare facility at least hourly. The learning inference model using a machine learning or deep learning algorithm is preferably configured to identify at least one milestone associated with a patient receiving care in a healthcare facility. Preferably, the learning inference model, using a machine learning or deep learning algorithm, is further configured to: update and track the at least one milestone twice a day until the patient's bed is ready for occupancy by another patient; generate a first alert within a predetermined time window before a selected milestone; and generate a second alert within a second predetermined time window after a target time for the selected milestone to occur has passed. Additionally, the learning inference model using a machine learning or deep learning algorithm is further configured to predict a volume of patients that will be staying in the healthcare facility for the at least two respective time periods per day based on the received flow data and the predicted length of stay for the patients, and determine staffing requirements for the at least two time periods a day based on the predicted volume of patients for the respective at least two time periods, wherein the at least two respective time periods per day are hourly time periods. In certain embodiments, generating a learning inference model using a machine learning or deep learning algorithm is contingent upon data captured from one or more external data sources relative to the PLM server 502, wherein PLM server 502 is configured to normalize the captured data for analysis, which may include usage of a Large Language Model (LLM) or Rule-Based Expert System.

Next, at step 804, the PLM server 502 preferably analyzes the captured data, using the generated learning inference model, to generate, using at least a portion of the captured data, one or more predictions regarding one or more conditions to occur in the future that are associated with one or more resources of the healthcare facility associated with the purpose of stay for the patient. For instance, in certain embodiments, the generated one or more predictions includes a length of stay for the patient for treatment of the medical condition. In other embodiments, the one or more conditions to occur in the future that are associated with one or more resources relate to a bed and/or room that is to be required for the patient's predicted length of stay, and wherein the generated one or more predictions also includes predicting staff required for treatment of the patient's medical condition. Additionally, the one or more conditions include particular staff members that are required for the patient's predicted length of stay. Other illustrated embodiments include one or more medical supplies needed for the patient for treatment of the patient's medical condition that are required for the patient's predicted length of stay (e.g., including medical supplies needed in an operating predicted to be required for the patient).

Next, at step 806, the PLM server 502 analyzes, using AI techniques, the one or more predictions for recommending an allocation of at least one of the one or more resources on the basis of the results, wherein recommending an allocation of at least one of the one or more resources is preferably determined based in part on analyzing historical data relating to resources required for the patient's purpose of stay. The allocation of at least one of the one or more resources may include allocation of a bed and/or room for the patient's predicted length of stay and may further include indication of whether the determined bed and/or room required for the patient's length of stay is available in the healthcare facility. Additionally, the allocation of at least one of the one or more resources may further include recommending whether to transfer one or more other patient's from a first healthcare unit to a second healthcare unit for making the bed and/or room available for the patient during the patient's predicted length of stay, which may further include recommending an allocation of at least one of the one or more resources includes allocation of staff members for the patient's predicted length of stay. It is to be appreciated and understood that the aforesaid allocation of one or more resources likewise encompasses reallocation of one or more resources in a healthcare facility. For instance, one or more healthcare staff personal (e.g., nurses) may be reallocated from a first healthcare unit to a second healthcare unit that is, or will be, understaffed relative to the first healthcare unit.

In accordance with certain illustrated embodiments, recommending an allocation of at least one or more resources by the PLM server 502 using AI techniques includes generating at least one recommendation of a number of staff to schedule for providing medical care for a time period in the future for patients in at least one healthcare unit of the healthcare facility based in part on a prediction of a number of patients expected to occupy the healthcare unit during a certain time period in the future. It is noted the number of staff to schedule may be determined based in part on analyzing historical data indicating an average patient to medical personnel ratio within the healthcare unit during a corresponding time period in the past.

In accordance with other illustrated embodiments, recommending an allocation of at least one of the one or more resources by the PLM server 502 using AI techniques includes allocation of the predicted one or more medical supplies required for the patient's predicted length of stay, which may include indication of whether the determined one or more medical supplies required for the patient's predicted length of stay are available in the healthcare facility (e.g., medical supplies predicted to be required in an operating room environment). Thus, the illustrated embodiments provide significant improvements in computer applications for determining one or more predicted requirements and/or events for a healthcare facility corresponding to a scheduled inflow of patients by using artificial intelligence and/or machine learning techniques.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An Artificial Intelligence (AI) based computer system for determining one or more predicted requirements for a healthcare facility corresponding to a scheduled inflow of patients, comprising:
a memory configured to store instructions;
a processor disposed in communication with the memory and coupled to a computer network, wherein the processor generates a learning inference model using a machine learning or deep learning algorithm configured to:
capture data, from the computer network, containing information relating to patient inflow to the healthcare facility wherein the data includes a purpose of stay for a patient;
analyze the captured data, using the generated learning inference model, to generate, using at least a portion of the captured data, one or more predictions regarding one or more conditions to occur in the future that are associated with one or more resources of the healthcare facility associated with the purpose of stay for the patient;
analyze the one or more predictions for recommending an allocation and/or reallocation of at least one of the one or more resources.

2. The AI based computer system as recited in claim 1, wherein recommending an allocation of at least one of the one or more resources is determined based in part on analyzing historical data relating to resources required for the patient's purpose of stay.

3. The AI based computer system as recited in claim 2, wherein the purpose of stay relates to treatment of a medical condition.

4. The AI based computer system as recited in claim 3, wherein the generated one or more predictions includes a length of stay for the patient for treatment of the medical condition.

5. The AI based computer system as recited in claim 4, wherein the one or more conditions to occur in the future that are associated with one or more resources relate to a bed and/or room that is to be required for the patient's predicted length of stay.

6. The AI based computer system as recited in claim 5, wherein recommending an allocation of at least one of the one or more resources includes allocation of a bed and/or room for the patient's predicted length of stay.

7. The AI based computer system as recited in claim 6, wherein recommending an allocation of at least one of the one or more resources further includes indication of whether the determined bed and/or room required for the patient's length of stay is available in the healthcare facility.

8. The AI based computer system as recited in claim 6, wherein recommending an allocation of at least one of the one or more resources further includes recommending whether to transfer one or more other patients from a first healthcare unit to a second healthcare unit for making the bed and/or room available for the patient during the patient's predicted length of stay.

9. The AI based computer system as recited in claim 3, wherein the generated one or more predictions includes predicting staff required for treatment of the patient's medical condition.

10. The AI based computer system as recited in claim 9, wherein the one or more conditions to occur in the future that are associated with one or more resources relate to particular staff members that are required for the patient's predicted length of stay.

11. The AI based computer system as recited in claim 10, wherein recommending an allocation of at least one of the one or more resources includes generating at least one recommendation of a number of staff to schedule for providing medical care for a time period in the future for patients in at least one healthcare unit of the healthcare facility is based in part on a prediction of a number of patients expected to occupy the healthcare unit during a certain time period in the future.

12. The AI based computer system as recited in claim 9, wherein the number of staff to schedule is determined based in part on analyzing historical data indicating an average patient to medical personnel ratio within the healthcare unit during a corresponding time period in the past.

13. The AI based computer system as recited in claim 4, wherein the learning inference model using a machine learning or deep learning algorithm is further configured to:
identify at least one milestone associated with a patient receiving care in a healthcare facility;
update and track the at least one milestone twice a day until the patient's bed is ready for occupancy by another patient;
generate a first alert within a predetermined time window before a selected milestone; and
generate a second alert within a second predetermined time window after a target time for the selected milestone to occur has passed.

14. The AI based computer system as recited in claim 13, wherein the learning inference model using a machine learning or deep learning algorithm is further configured to:
predict a volume of patients that will be staying in the healthcare facility for the at least two respective time periods per day based on the received flow data and the predicted length of stay for the patients; and
determine staffing requirements for the at least two time periods a day based on the predicted volume of patients for the respective at least two time periods.

15. The AI based computer system as recited in claim 1, wherein generating a learning inference model using a machine learning or deep learning algorithm is contingent upon data captured from one or more external data sources.

16. The AI based computer system as recited in claim 22, wherein the processor is further configured to utilize a Large Language Model (LLM) for recommending an allocation and/or reallocation of at least one of the one or more resources.

17. An Artificial Intelligence (AI) computer method for determining one or more predicted requirements for a healthcare facility corresponding to a scheduled inflow of patients, comprising:
capturing data, from the computer network, containing information relating to patient inflow to the healthcare facility wherein the data includes a purpose of stay for a patient;
analyzing the captured data, using the generated learning inference model, to generate, using at least a portion of the captured data, one or more predictions regarding one or more conditions to occur in the future that are associated with one or more resources of the healthcare facility associated with the purpose of stay for the patient; and
analyzing the one or more predictions for recommending an allocation and/or reallocation of at least one of the one or more resources.

18. The AI computer method as recited in claim 17, wherein generating a learning inference model using a machine learning or deep learning algorithm is contingent upon data captured from one or more external data sources.

19. The AI computer method as recited in claim 18, wherein the processor is further configured to utilize a Large Language Model (LLM) for recommending an allocation and/or reallocation of at least one of the one or more resources.

20. The AI computer method as recited in claim 19, wherein recommending an allocation of at least one of the one or more resources is determined based in part on analyzing historical data relating to resources required for the patient's purpose of stay as extracted from a computer database via a computer network.
